(19) United States
(12) Patent Application Publication
(10) Pub. No.: US 2026/0060223 A1
(43) Pub. Date: Mar. 5, 2026

(54) WORM BED SYSTEM AND APPARATUS

(71) Applicant: Annelida Soil Solutions Ltd., Nisku (CA)

(72) Inventors: Hai Le, Edmonton (CA); Michael Launer, Spruce Grove (CA); Jamie DePape, Spruce Grove (CA)

(21) Appl. No.: 19/318,357

(22) Filed: Sep. 3, 2025

Related U.S. Application Data
(60) Provisional application No. 63/690,232, filed on Sep. 3, 2024.

(51) Int. Cl. A01K 67/33

(52) U.S. Cl. CPC A01K 67/33

(57) ABSTRACT

A method and apparatus for distributing organic matter to a stack of worm bed reactors, where each worm bed reactor has a receptacle with a screened bottom surface, sidewalls, an open top, and is filled with substrate and worms. A distribution gantry moveable from a docking area along the length of each reactor. An organic matter feeder overlies the docking area of each stack and supplies organic matter to each gantry in the stack. The gantries travel along the reactor to distribute the organic matter.

### TECHNICAL FIELD

This relates to the processing of organic matter, and in particular, to a system and method of processing organic matter using worm bed reactors.

### BACKGROUND

Organic matter can be processed by breaking down the organic material to generate a usable end product. One method to process such organic matter is through the use of vermiculture, in which worms break down the organic matter and generate the end product in the form of castings. United States Patent Application Publication No. 2002/012b4658 (Holcombe et al.) entitled “Solids Discharge Device” teaches a composting bin for use in vermiculture.

### SUMMARY

According to an aspect, there is provided a method of distributing organic matter to worm bed reactors, the method comprising the steps of providing one or more stacks of worm bed reactors, each stack of worm bed reactors comprising at least an upper worm bed reactor overlying a lower worm bed reactor, each worm bed reactor having a length and comprising a substrate receptacle having a screened bottom surface, sidewalls, and an open top, the screened bottom surface permitting loosened castings to fall from the worm bed reactor, filling each worm bed reactor with substrate and worms, providing a distribution system comprising a plurality of tracks, each track extending from a docking area at a first end of the stack of worm bed reactors along the length of each worm bed reactor, a distribution gantry mounted to the track above each worm bed reactor, and an organic matter lifting mechanism overlying the docking area of each stack of worm bed reactors, and supplying organic matter to each of the worm bed reactors by using the organic matter lifting mechanism, depositing organic matter in the distribution gantry of the upper worm bed reactor in the docking area, causing the distribution gantry of each upper worm bed reactor to travel along the length of the respective upper worm bed reactor along the track of the respective upper worm bed reactor, and once the distribution gantry of the upper worm bed reactor has moved sufficiently along the length of each upper worm bed reactor to exit the docking area, depositing organic matter in the distribution gantry of the lower worm bed reactor and causing the distribution gantry of the lower worm bed reactor to travel the length of the lower worm bed reactor along the track of the lower worm bed reactor.

According to other aspects, the method may further comprise the step of monitoring conditions in each worm bed reactor using sensors carried by the distribution gantries as the distribution gantries move along the track, the organic matter lifting mechanism may be a lifting bucket system vertically moveable through the docking area and laterally moveable between adjacent docking areas, the organic matter lifting mechanism may be an auger arrangement that extends across the docking areas, each stack of worm bed reactors may further comprise one or more additional worm bed reactors underlying the lower worm bed reactor, and the method may further comprise the step of depositing organic matter into each additional worm bed reactor once the distribution gantry immediately above each additional worm bed reactor has moved sufficiently, and the track may be a set of rails.

According to an aspect, there is provided an apparatus for processing organic matter, the apparatus comprising one or more stacks of worm bed reactors, each stack of worm bed reactors comprising at least an upper worm bed reactor overlying a lower worm bed reactor, each worm bed reactor having a length and comprising a substrate receptacle having a screened bottom surface, sidewalls, and an open top, the screened bottom surface permitting loosened material to fall from the worm bed reactor, a distribution system comprising tracks that extend from a docking area along the length of each worm bed reactor, and a distribution gantry positioned on the track above each worm bed reactor, an organic matter delivery system comprising a lifting mechanism overlying the docking area of each stack of worm bed reactors and an opening in the docking area above each distribution gantry to allow the lifting mechanism to deposit organic matter, a cutting mechanism placed within each worm bed reactor for cutting a bottom portion of the substrate to loosen the bottom portion of the substrate and cause it to fall through the screened bottom surface, and a castings collecting apparatus underlying each worm bed reactor for collecting the bottom portion of the substrate after falling through the screened bottom surface.

According to other aspects, the distribution system may comprise sensors for monitoring conditions in each worm bed reactor, the apparatus may further comprise a worm removing mechanism that removes worms from one or more worm bed reactors, and may further comprise a worm conveyor for conveying the worms removed by the worm removing mechanism from the worm bed reactor to a collection location, the lifting mechanism may be a lifting bucket system vertically moveable through the docking area and laterally moveable between adjacent docking areas or an auger arrangement, the lifting bucket system may have a lifting bucket comprising a sidewall and a bottom surface, and the bottom surface may comprise a set of louvres that selectively open to deposit organic matter, the castings collecting apparatus may comprise a conveyor that moves along the worm bed reactor to transport the bottom portion of the substrate to a collection location, the apparatus may further comprise a hopper at the collection location, the conveyor transporting the bottom portion of the substrate into the hopper, the hopper being connected to a vacuum conveying line to transport the bottom portion of the substrate to a processing location, the processing location may comprise a separator for separating a fine portion of the bottom portion of the substrate, or the castings collecting apparatus may comprise an auger system configured to transport the bottom portion of the substrate to a collection location, each stack of worm bed reactors may further comprise one or more additional worm bed reactors underlying the lower worm bed reactor, and the track may comprise a set of rails.

According to an aspect, there is provided a gantry system for delivering organic matter to a worm bed reactor, the worm bed reactor comprising a length and a width, the gantry system comprising a track extending along the length of the worm bed reactor, and a distribution gantry mounted to the track, the distribution gantry comprising track engagements sized to engage with and move along the track, and an organic matter receptacle having a sidewall surrounding an open bottom that extends substantially across the width of the worm bed reactor, and a conveyor below the open bottom that extends across the open bottom, the conveyor receiving organic matter from the open bottom and moving the organic matter from the within the organic matter receptacle to the worm bed reactor along the length of the worm bed reactor as the distribution gantry moves along the track.

According to other aspects, the organic matter receptacle may comprise internal baffles positioned across the open bottom, the conveyor belt may move across the open bottom at the same rate as the rail engagements, the distribution gantry may be self-propelled along the track, the movement of the distribution gantry may be controlled by a remote controller, the distribution gantry may be programmed to be fully automated, the distribution gantry may carry batteries, and the batteries may be charged by a docking station at a first end of the track, the docking station may be solar powered, the distribution gantry may comprise volume sensors that detect the volume of organic matter contained within the organic matter receptacle, the distribution gantry may return to a docking area to be refilled when empty, the distribution gantry may comprise sensors that measure one or more of temperature, pH, heat signatures, moisture content, worm concentration, and worm bed mass in the distribution gantry, the worm bed reactor, or both the distribution gantry and the worm bed reactor, and the tracks may comprise a set of rails, and the track engagements may comprise rail engagements.

In other aspects, the features described above may be combined together in any reasonable combination as will be recognized by those skilled in the art.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is an end schematic view of a lifting bucket method for loading distribution gantries.

FIG. 2 is an end schematic view of an auger method for loading distribution gantries.

FIG. 3 is a side schematic view of an apparatus for processing organic matter.

FIG. 4 is a perspective view of a distribution gantry over a cut away portion of a worm bed.

FIG. 5 is a side elevation view of an alternative distribution system.

FIG. 6 is top plan view of the alternative distribution system of FIG. 5.

FIG. 7 is a perspective view of an organic matter receptacle.

FIG. 8 is a side elevation view in cross section of a lifting bucket in a closed position.

FIG. 9 is a side elevation view in cross section of a lifting bucket in an opened position.

FIG. 10 is a top perspective view of a lifting bucket in a closed position.

FIG. 11 is a bottom perspective view of a lifting bucket in a closed position.

FIG. 12 is a photograph of a front of a distribution gantry.

FIG. 13 is a perspective schematic view of an apparatus for processing organic matter that includes a vacuum collection system.

FIG. 14 is a side elevation view of a hopper for a vacuum collection system.

FIG. 15 is a side elevation view in cross-section of a hopper for a vacuum collection system.

FIG. 16 is a top plan view of a hopper for a vacuum collection system.

FIG. 17 is an end elevation view of an apparatus for processing organic matter.

### DETAILED DESCRIPTION OF PREFERRED EMBODIMENTS

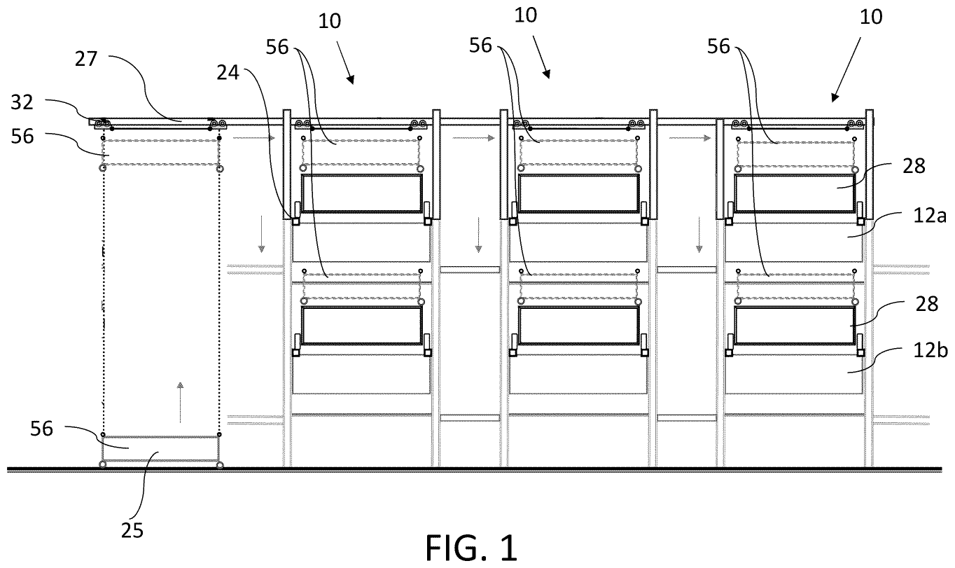

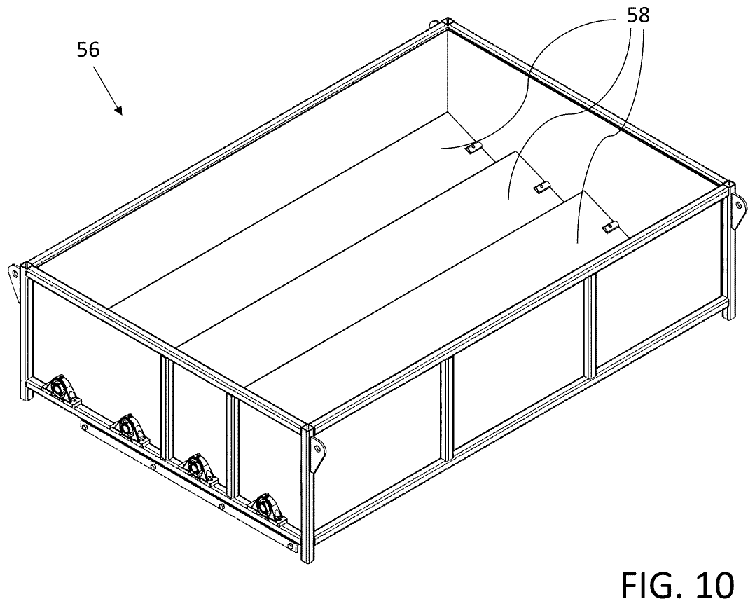

An apparatus for processing organic matter will now be described with reference to FIG. 1 through 17.

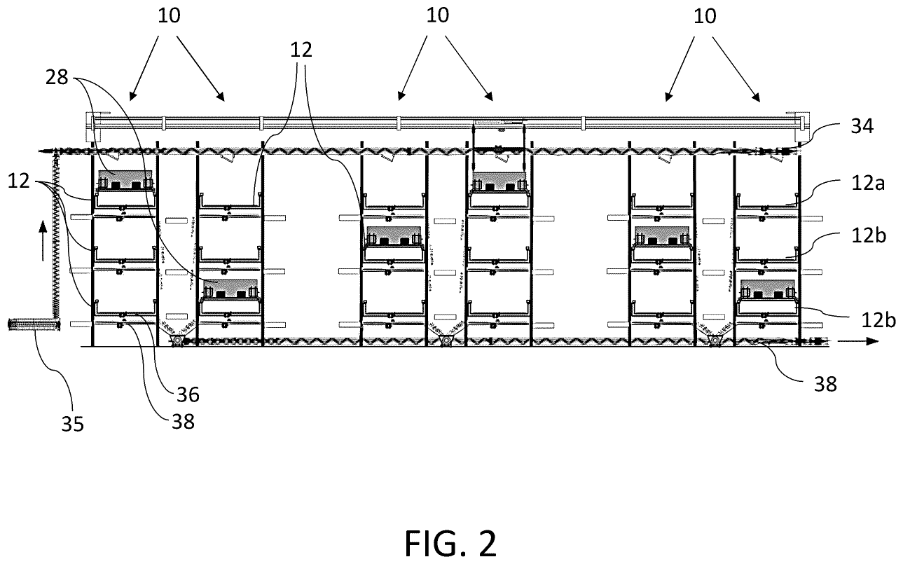

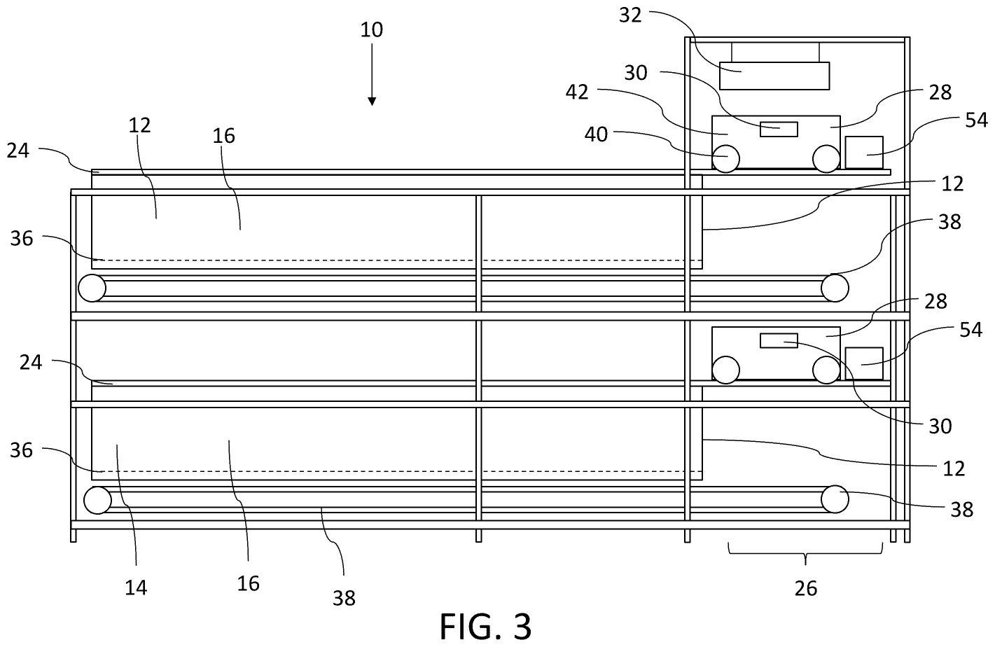

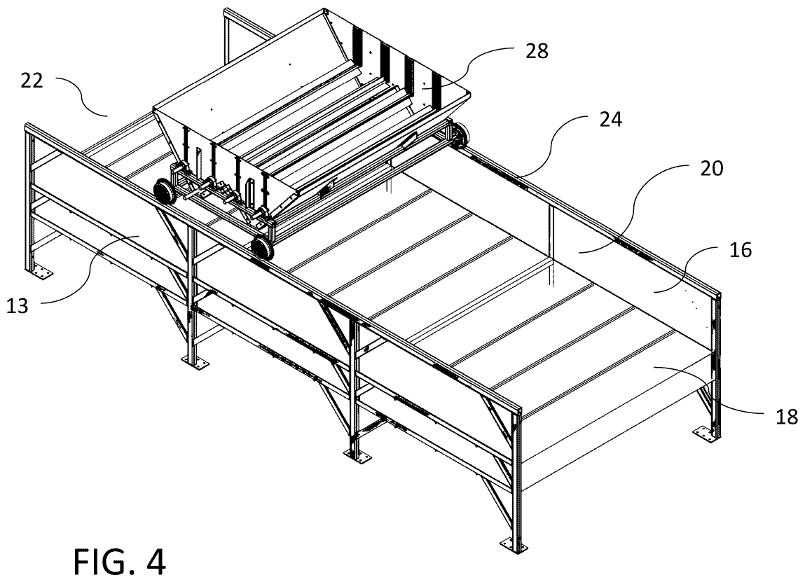

Referring to FIG. 1 and FIG. 2, an apparatus for processing organic matter is shown with stacks 10 of worm bed reactors 12. There may be a single stack 10 or multiple stacks 10 of worm bed reactors 12, and each stack 10 may have two or more worm bed reactors, depending on space constraints and desires specifications, including an upper worm bed reactor 12a at the top of stack 10, and one or more lower worm bed reactors 12b underlying upper worm bed reactor. It will also be understood that the stack 10 of worm bed reactors 12 may be any number of worm bed reactors 12 high, and may, for example, include one or more additional underlying worm bed reactors 15 underlying lower worm bed reactor 12b, as shown in FIG. 2. Referring to FIG. 4, each worm bed reactor 12 has a screened bottom surface 18 surrounded by sidewalls 20 that define a substrate receptacle 16 and an open top 22. Screened bottom surface 18 may, for example, be a metal mesh, a surface with apertures, or the like, provided that a sufficient amount of the substrate is permitted to pass through screened bottom surface 18 when harvesting the castings as discussed below. Worm bed reactors 12 may be provided with a cover (not shown) that may be removed as needed to cover the top surface of the substrate to retain moisture and prevent contamination, while being retractable to allow additional organic material to be added. Referring to FIG. 3, a distribution system is connected to each worm bed reactor 12, the distribution system being made up of rails 24, or other suitable type of track as will be recognized by those skilled in the art, that extend from a docking area 26 spaced from each worm bed reactor 12 along the length of each worm bed reactor 12, and a distribution gantry 28 positioned on the rails 24 above each worm bed reactor 12. The distribution system may include sensors 30 for monitoring conditions in each worm bed reactor 12. As shown, these sensors may be positioned on distribution gantry 28 and may collect sensor readings as distribution gantry 28 moves along rails 24. These sensors may also be placed within the worm bed reactor 12, or at other locations as needed in the particular application. For example, sensors 30 may detect chemicals or gases, the temperature within worm bed reactors 12, the volume or depth of substrate in worm bed reactors, etc. Sensors 30 may also capture and transmit live images of worm bed reactors 12 for an operator to monitor. There may be other structures, such as walkways, ladders, steps, etc. adjacent to or surrounding stacks 10 that allow workers to service, repair, test, etc. worm bed reactors 12.

The worms contained in the worm bed reactors 12 feed on organic matter, as well as consuming microorganisms carried by the organic matter, which may include harmful bacteria and other pathogenic microbes. The worms then produce castings, which are collected as a usable end product for applications such as acting as a substrate for growing plants. In the system described herein, the material being deposited into worm bed reactors is organic matter, while the material in the worm bed reactor may be referred to as a substrate, where the substrate may include a bottom layer that has been fully processed by the worms, i.e., castings, and an upper layer that will be at different stages of being processed by the worms.

In order to supply worm bed reactors 12 with organic matter, an organic matter delivery system is provided. The organic matter delivery system has a feeder, or a lifting mechanism, overlying docking area 26 of each stack 10 of worm bed reactors 12, and an opening in docking area 26 above each distribution gantry 28 that is positioned to allow the lifting mechanism to deposit organic matter. Referring to FIG. 1 and FIG. 3, the feeder includes a lifting bucket 32. In this embodiment, lifting bucket 32 is provided and an overhead crane is used to lift the bucket. The bucket is filled on ground level and then lifted and moved laterally to the docking area where organic matter is deposited into a desired distribution gantry 28. Referring to FIG. 2, the feeder is shown as being an auger arrangement 34. In this embodiment, organic matter may be introduced into auger 34 at ground level via a hopper 35, and auger 34 then lifts the organic matter to deposit into a desired distribution gantry 28. The input of auger 34 may be attached to a mixer. As the organic material below the top surface is consumed, the worms will tend to congregate near the top surface of the substrate contained in the worm bed reactor 12, and the castings resulting from the worms consuming the organic material will be at the bottom of the worm bed reactor 12. Referring to FIG. 3, a cutting or loosening mechanism 36 that is pulled through the worm bed reactor 12 to loosen or separate the bottom layer of substrate, is placed within each worm bed reactor 12. This may, for example, be a cutter bar or plough that is positioned within worm bed reactor 12. Cutting mechanism 36 may be attached to a driver on either end of worm bed reactor 12 that pulls cutting mechanism 36 through the substrate, such as by using chains or cables positioned beside or within worm bed reactor 12. The cutting mechanism 36 causes a bottom portion of the substrate within the worm bed reactor 12 to be loosened or dislodged, allowing it to fall through the apertures of the apertured bottom surface 18 of the worm bed reactor 12. The frequency with which the bottom portion is cut will depend on the height of the bottom casting portion, as well as the rate at which the organic matter is processed.

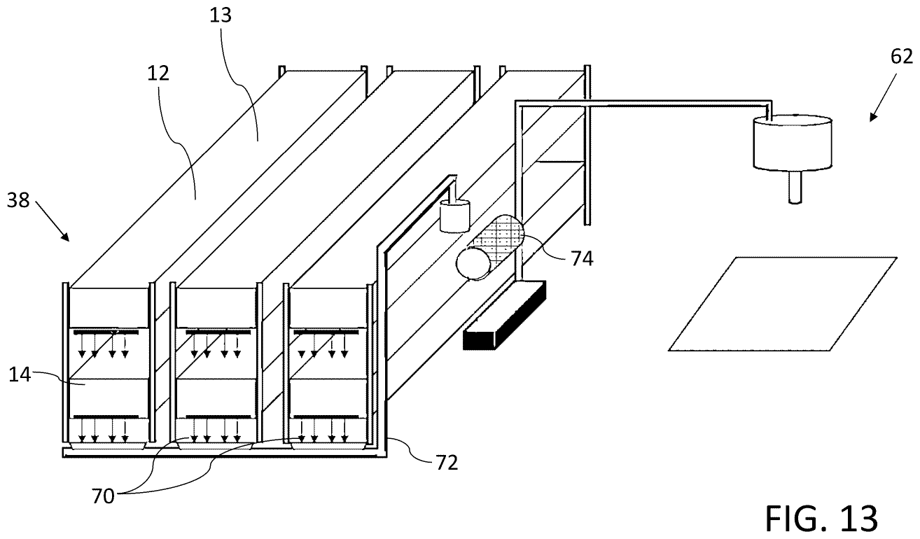

A castings collecting apparatus 38 underlies each worm bed reactor 12 for collecting the bottom portion of the substrate after it is cut. Each worm bed reactor 12 may be provided with hanging deflectors on the underside that deflect the falling material to ensure collection on the castings collecting apparatus 38. As shown in FIG. 3, the casting collecting apparatus 38 may be a conveyor belt that transports the collected substrate to a collection location. The conveyor belt may move the substrate to one or both ends of worm bed reactor 12 or may move the collected substrate to the side of worm bed reactors 12 to be collected. As shown in FIG. 2, the casting collecting apparatus 38 may also use an auger system configured to transport the collected substrate. As shown in FIG. 13 through FIG. 16, the casting collecting apparatus may include a vacuum collection system.

In the event that the worm concentration within the worm bed reactor 12 becomes too high, it may be desirable to provide a worm removing mechanism to remove worms from the one or more worm bed reactors 12, either to be moved to a different worm bed reactor 12, or as an end product. The apparatus may be provided with a worm conveyor for removing a top layer of material in each worm bed reactor and conveying the worms to a collection location (not shown).

Referring to FIG. 13, casting collection apparatus 38 may transport the bottom portion of the substrate to a collection and packaging system 62. The vacuum collection system may have a plurality of hoppers 70 placed at an end of the stacked worm bed reactors 12 that receive the collected bottom portion at the end of a conveyor or other collecting apparatus 38. In the depicted example, each stack of worm bed reactors 10 has one hopper 70 which receives collected substrate from both the upper and lower worm bed reactors 12 and 12b in the stack 10. The hoppers 70 feed collected substrate into a vacuum conveying line 72 that is connected to the bottom of each hopper 70 and transports the substrate to be processed further. A single vacuum conveying line 72 may have any number of hoppers 70 connected to it, or multiple vacuum lines may be used.

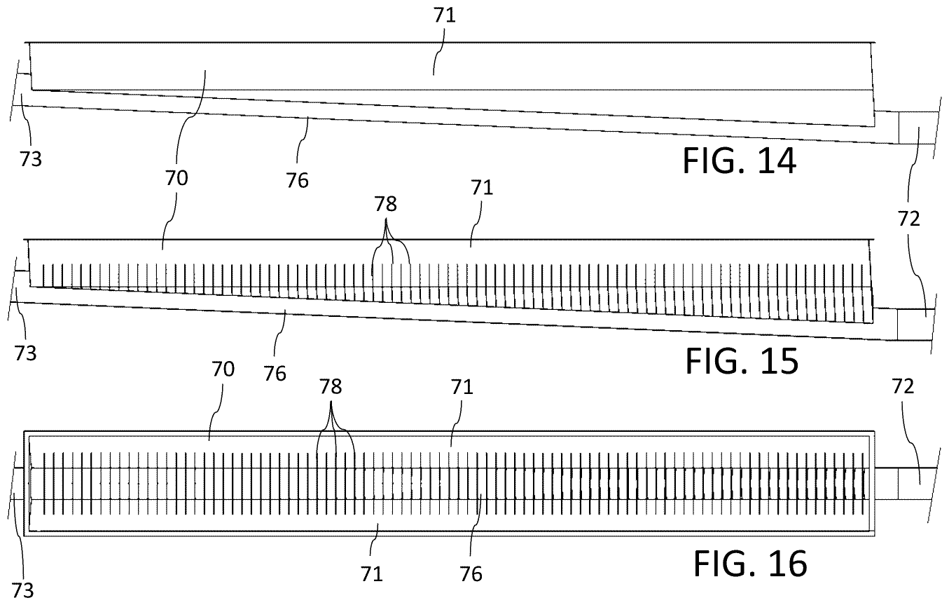

One example of a hopper 70 is shown in FIG. 14 to FIG. 16. Hopper 70 may have tapered sides 71 sized to catch all of the collected substrate from casting collection apparatus 38. Tapered sides 71 may direct the collected substrate to an inclined bottom pipe 76 that is connected to vacuum conveying line 72. Inclined bottom pipe 76 may have an adjustable angle of inclination to aid in the transportation of the collected substrate. A vibrator (not shown) may be attached to the hopper 70 to support the flow of collected substrate to the vacuum line 72. A vacuum is applied to the vacuum conveying line 72 and hopper 70 such that the collected substrate is urged toward the lower end of vacuum conveying line 72 by both gravity and airflow from an upper end 73 of hopper 70 and vacuum conveying line 72. The vacuum may be connected to the lower end of bottom pipe 76, while the higher end may provide either ambient air or positive pneumatic air pressure to assist flow in the pipe. One or more hoppers may be connected in series, with the bottom pipes 76 of each in fluid communication. Upper end 73 may have a valve (not shown) that may control an airflow rate through bottom pipe 76. Hopper 70 may have a plurality of baffles 78 or guiding plates spaced within hopper 70 that assist with separating the collected substrate and directing airflow into the bottom pipe 76. Baffles 78 may serve to reduce particulate clumping and prevent blockage of hopper 70 or conveying line 72.

The collected substrate may be passed from the first vacuum conveying system into other apparatus for further processing. For example, the collected substrate may be further separated and processed after passing through hopper 70, being transferred through a processing stage to a second vacuum system. A fine casting portion may be produced by reducing the particulate size or may be separated from the collected bottom portion of the substrate, such as by using a trommel 74, as shown in FIG. 13. The size of fine substrate collected may be controlled by the size of the mesh or holes in trommel 74. The fine portion of the casting that is collected from trommel 74 may be directed to a packaging area 62 by an additional vacuum system.

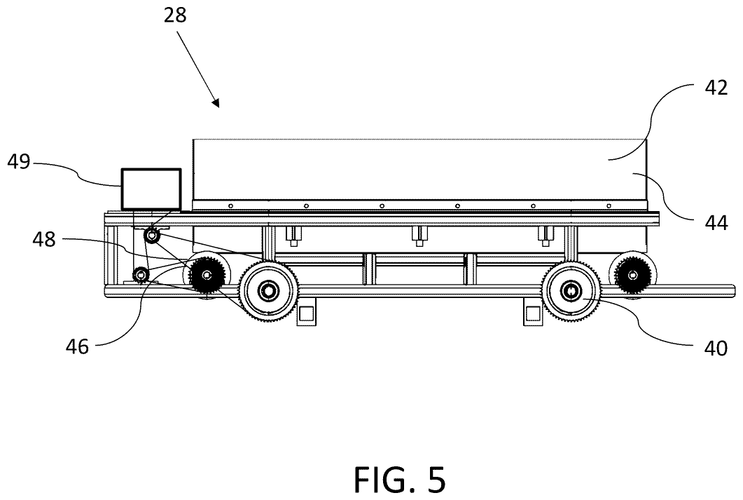

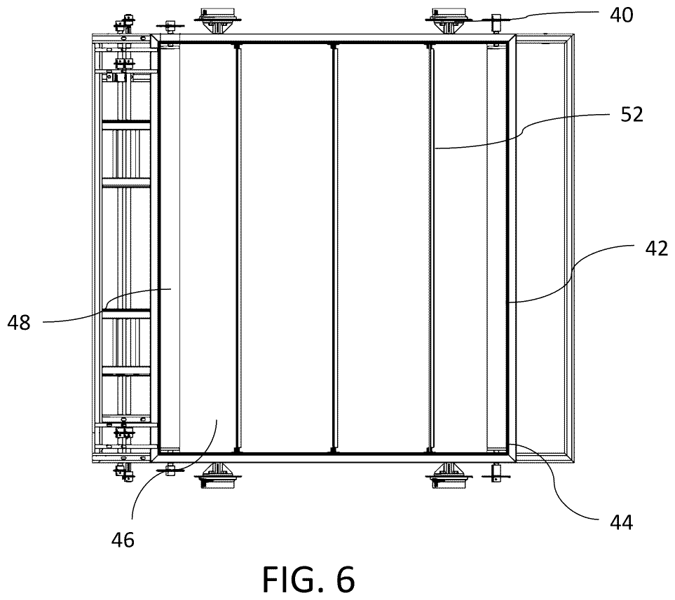

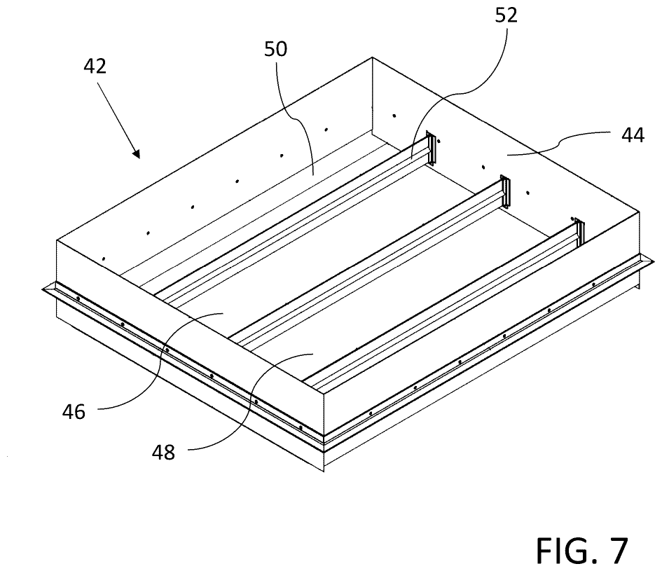

A gantry system for delivering organic matter to a worm bed reactor 12 will now be described. Worm bed reactor 12 has a length, a width, and an open top. The gantry system is made up of rails 24 extending along the length of the worm bed reactor 12, and distribution gantry 28 positioned on the rails. Distribution gantry 28 is configured to move along rails 24 and disburse the organic matter along the length of worm bed reactor 12. Referring to FIG. 4, a first embodiment of a distribution gantry 28 is shown. In this embodiment, distribution gantry 28 has sloped sides and internal paddles to direct organic matter out of distribution gantry 28. Referring to FIG. 5, an alternative embodiment for distribution gantry 28 is shown. In this embodiment, distribution gantry 28 is made up of rail engagements 40 that are sized to engage with and move along rails 24, and an organic matter receptacle 42. As shown in FIG. 6 and FIG. 7, organic matter receptacle 42 has a sidewall, such as rectangular sides 44 extending above rail engagements 40 and a conveyor 46 placed below rectangular sides 44 and defining an open bottom of organic matter receptacle 42. Conveyor 46 has a belt 48 that moves along the bottom of organic matter receptacle 42, and an opening 50 at the front of distribution gantry 28 through which organic matter may be urged by the movement of the conveyor belt 48 to exit the distribution gantry and deposited into worm bed reactor 12. Organic matter receptacle 42 may also contain internal baffles 52 to spread and direct organic matter as it moves within organic matter receptacle 42.

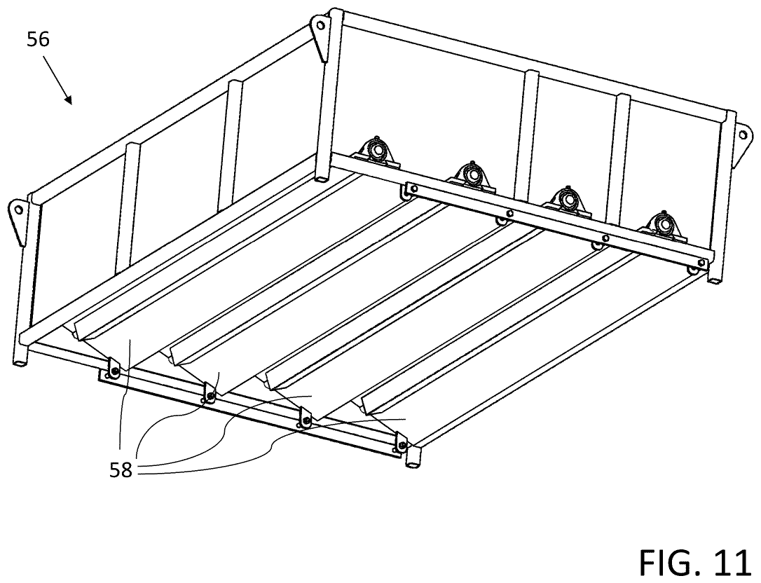

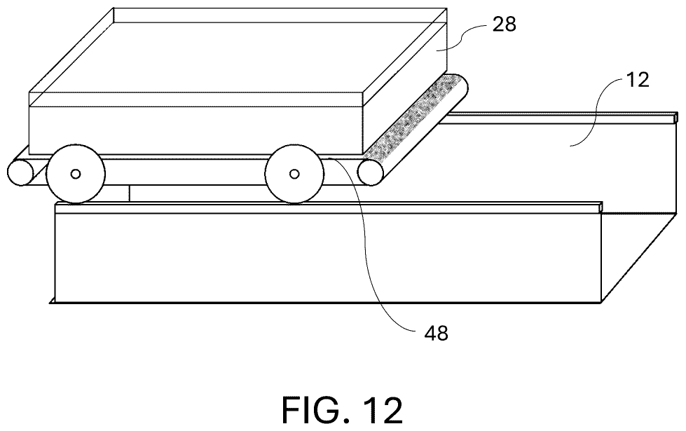

Referring to FIG. 5, conveyor belt 48 rotates around conveyor 46 as distribution gantry 28 moves along rails 24 and causes the organic matter contained within organic matter receptacle 42 to be spread into the open top of worm bed reactor 12. The rotation of conveyor belt 48 may be tied to the movement of distribution gantry 28 along rails 24, and may use a single mechanism, such that the rotation of conveyor belt 48 directly proportional to the speed of forward movement of distribution gantry 28, such as where conveyor belt 48 is rotated at the same rate as rail engagements 40. A more detailed example of gantry 28 with conveyor belt 48 is shown in FIG. 12. These rotation rates may also be separately calibrated and may either use the same or separate power sources. The rotation rates may be optimized for the particular composition of the organic matter being delivered. Distribution gantry 28 may have a drive 49, such as an electric motor with onboard, rechargeable batteries, which allows gantry 28 to be self-propelled. Distribution gantry 28 may return to a docking station 54 at a first end of rails 24 in order to charge the batteries, as shown in FIG. 3. Docking station 54 may be solar powered or may use other renewable energy sources. The distribution gantry 28 may be controlled by a remote controller operated by a worker or may be fully programmed to work automatically following a predetermined schedule. For example, the distribution gantry may be programmed to operate on a daily feeding schedule.

Distribution gantry 28 may carry sensors or scanners 30, which may include internal volume sensors that detect the volume of organic matter contained within organic matter receptacle 42. Distribution gantry 28 may be configured to monitor the volume of organic matter and return to docking area 26 when empty in order to be refilled with organic matter. Sensors or scanners 30 may also include sensors for monitoring conditions within worm bed reactor 12 as distribution gantry 28 moves along its length. For example, sensors or scanners 30 may measure one or more of temperature, pH, heat signatures, moisture content, worm concentration, and worm bed mass. The conditions monitored may generally relate to the biological systems, the worm population, the casing quantity, and system diagnostics for each worm bed. Sensors or scanners used to monitor these conditions may use heat signatures, such as through the use of infrared scanning, or imaging, such as sonar, to determine worm concentration in each bed. These sensors may also be used to determine the harvestable casting quantity in each bed. This information may be used to generate a three-dimensional image of the worm bed mass for improvement planning and may also be used to determine if worms should be removed from the bed. For example, it may be beneficial to detect worm concentration and collect a portion of the worms before population numbers reach a level at which worms slow or stop reproduction. This may allow for the maximum number of worms to be produced. Sensors may also determine if a harmful contaminate or other condition is causing worms to avoid specific areas of worm bed reactor 12 and allow this condition to be remediated. Distribution gantry 28 may also carry other apparatus, such as audible alarms and strobe lights to indicate positioning of each distribution gantry 28 to working personnel or may carry position sensors as part of a real-time positioning system to record the exact location of gantry 28 on the worm bed reactor 12 at any given moment in time.

In order to collect and interpret the data from sensors 30, and/or to control the movement of distribution gantry 28, a processor (not shown) may be provided. This may be any suitable processor, such as a general-purpose computer, with suitable programming and software that allows for the acquisition and interpretation of the data, and to control distribution gantry 28 accordingly.

A method of distributing organic matter to worm bed reactors 12 will now be described. One or more stacks 10 of worm bed reactors 12 are provided, each stack having at least upper worm bed reactor 12 overlying lower worm bed reactor 12b. Each worm bed reactor 12 has substrate receptacle 16 with apertured or screened bottom surface 18, sidewalls 20, and open top 22. Each worm bed reactor 12 is filled with both substrate and worms. Distribution gantry 28 is provided on rails 24 above each worm bed reactor 12. Organic matter is periodically supplied to each worm bed reactor 12 by positioning each distribution gantry 28 in docking area 26 of each stack 10 of worm bed reactors 12. An organic matter feeder such as a hoist and trolley system 32 or auger arrangement 34 is provided, which overlies docking area 26 of each stack of worm bed reactors 12. Organic matter is deposited in distribution gantry 28 of each upper worm bed reactor 12 of each stack 10 of worm bed reactors 12. Distribution gantry 28 of upper worm bed reactor 12 is then caused to travel the length of upper worm bed reactor 12 along rails 24. Once distribution gantry 28 of upper worm bed reactor 12 has moved sufficiently along the length of upper worm bed reactor 12 to exit docking area 26, organic matter may then be deposited in distribution gantry 28 of lower worm bed reactor 12b. Distribution gantry 28 of lower worm bed reactor 12b may then be caused to travel the length of worm bed reactor 12b along rails 24, and after it exits docking area 26, any additional underlying distribution gantries 28 may be successively supplied with organic matter and caused to travel along rails 24 in turn. As discussed above, distribution gantry 28 may monitor conditions in each worm bed reactor 12 using sensors or scanners 30 as it moves along rails 24.

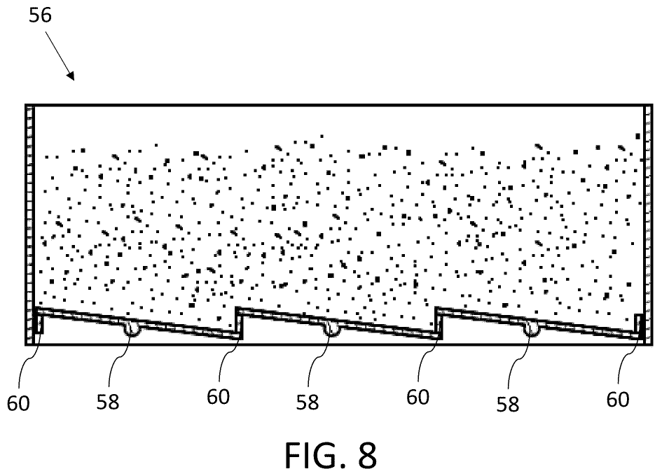

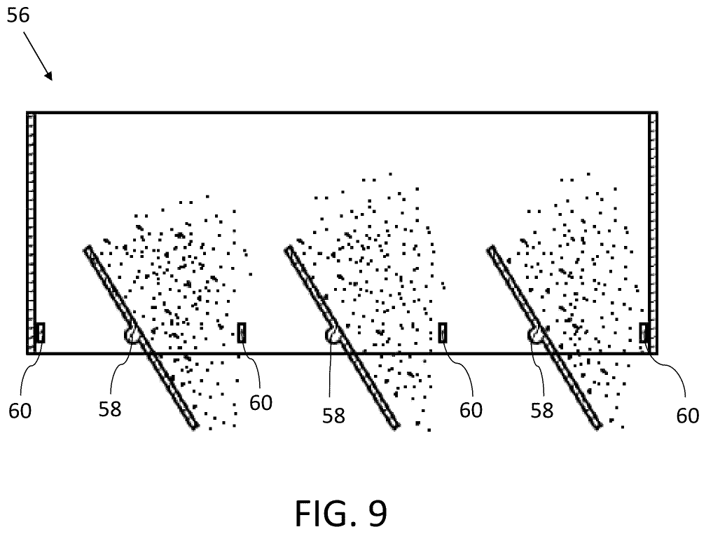

Referring to FIG. 8 through FIG. 11, one embodiment of a bucket 56 for use with hoist and trolley 32 as described above is shown. As shown in FIG. 8, the bottom of bucket 56 is formed by a series of pivoting surfaces 58 held against stops 60. Pivoting surfaces 58 may be connected to an actuator, such as a hydraulic cylinder or an electrical linear actuator that pivots surfaces 58 from the close position to the opened position, as shown in FIG. 9. This structure may reduce the force required by the opening mechanism to open the bin, as the angle of the pivoting surface 58 is such that leverage is applied by the organic matter to pivoting surface 58. This may reduce the time or energy required to open bucket 56 to deliver organic matter to each distribution gantry 28. This may also reduce the required operating air clearance above the distribution gantry 28. Pivoting surfaces 58 and stops 60 may form a set of louvres across the bottom surface that selectively open to deposit organic matter.

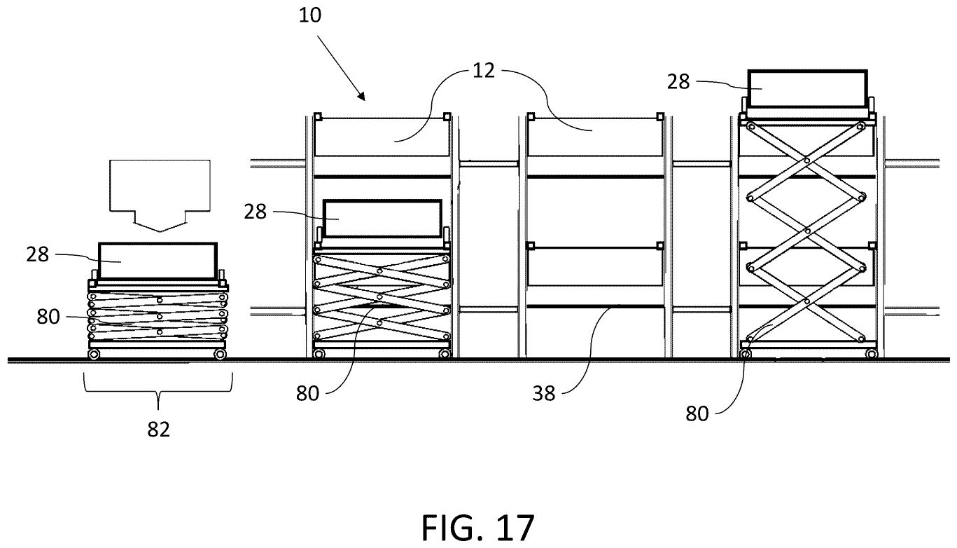

In another embodiment, referring to FIG. 17, instead of a feeder as described above, a movable docking station may be employed. For example, one docking station may be provided to serve all worm bed reactors 12 in a stack 10, and the docking station may be capable of both lateral and vertical movement, such as a mobile scissor lift, which is able to lift the gantry 28 to each worm bed reactor 12. This docking station may carry distribution gantry 28 to an fill location to fill distribution gantry 28 with organic matter and then carry gantry 28 to each worm bed reactor 12. The movable docking station may be calibrated to align the distribution gantry 28 with the rails associated with each worm bed reactor 12. Once the distribution gantry 28 has delivered all of the organic matter to the worm bed reactor, it may return to the movable docking station to be carried back to the uploading location and then be carried to the next worm bed reactor 12. In this manner, it may be possible to employ a single distribution gantry and docking station rather than a separate lifting mechanism to deliver organic matter to multiple distribution gantries.

In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be one and only one of the elements.

The scope of the following claims should not be limited by the preferred embodiments set forth in the examples above and in the drawings but should be given the broadest interpretation consistent with the description as a whole.

What is claimed is:

1. A method of distributing organic matter to worm bed reactors being vertically arranged in a stack, each of the worm bed reactors having a screened bottom surface that permits loosened material to fall from the worm bed reactor and a track that extends above the worm bed reactor, the method comprising the steps of:
introducing worms into each worm bed reactors;
supplying organic matter to each worm bed reactor using an organic matter distribution system, the organic matter distribution system comprising:
a docking area at a first end of the stack;
a distribution gantry mounted to the track above each worm bed reactor, the distribution gantry being adapted to distribute particulate matter while moving along the track; and
a feeder for filling the distribution gantry of each worm bed reactor with organic matter;
wherein supplying organic matter to each worm bed reactor comprises:
with a selected distribution gantry in the docking area adjacent to a selected worm bed reactor in the stack, depositing organic matter from the feeder into the selected distribution gantry;
causing the selected distribution gantry to move from the docking area along the track of the selected worm bed reactor while distributing the organic matter;
with a further distribution gantry in the docking area adjacent to a further worm bed reactor in the stack, depositing organic matter from the feeder into the selected distribution gantry; and
causing the further distribution gantry to move from the docking area along the track of the further worm bed reactor while distributing the organic matter.

2. The method of claim 1, wherein the docking area is vertically aligned adjacent to each worm bed reactor and in communication with each track.

3. The method of claim 2, wherein organic matter is deposited from the feeder into the selected distribution gantry docking area after the selected distribution gantry moves from the docking area.

4. The method of claim 1, wherein the feeder is a lifting bucket that is vertically moveable through the docking area.

5. The method of claim 4, further comprising more than one stack of worm bed reactors and wherein the feeder is laterally moveable between the more than one stacks of worm bed reactors.

6. The method of claim 4, further comprising the step of depositing organic matter into the feeder using an organic matter conveyor having an inlet in communication with a source of organic matter and an outlet in communication with the docking area.

7. The method of claim 1, further comprising the step of loosening material adjacent to the screened bottom such that the loosened material falls through the screened bottom.

8. An apparatus for processing organic matter, the apparatus comprising:
worm bed reactors being vertically arranged in a stack, each of the worm bed reactors having a screened bottom surface that permits loosened substrate to fall from the worm bed reactor and a track that extends above the worm bed reactor;
an organic matter distribution system comprising:
a docking area at a first end of the stack;
a distribution gantry mounted to the track above each worm bed reactor, the distribution gantry being adapted to distribute organic matter while moving along the track; and
a feeder for filling the distribution gantry of each worm bed reactor with organic matter;
a cutter bar within each worm bed reactor, the cutter bar being moveable in a direction parallel to the screened bottom surface, the cutter bar being adapted to dislodge a bottom portion of the substrate in the worm bed reactor and cause the bottom portion to fall through the screened bottom surface; and
a substrate collector underlying each worm bed reactor and disposed to collect the bottom portion of the substrate after falling through the screened bottom surface.

9. The apparatus of claim 8, wherein the docking area is vertically aligned adjacent to each worm bed reactor and in communication with each track.

10. The apparatus of claim 9, wherein the distribution gantries are moveable from the docking area to permit vertically spaced distribution gantries to be filled sequentially by the feeder.

11. The apparatus of claim 8, wherein the feeder is a lifting bucket that is vertically moveable through the docking area.

12. The apparatus of claim 11, further comprising more than one stack of worm bed reactors and wherein the feeder is laterally moveable between the more than one stacks of worm bed reactors.

13. The apparatus of claim 11, wherein the lifting bucket comprising a sidewall and bottom surface, the bottom surface comprising a set of louvres that selectively open to deposit organic matter.

14. The apparatus of claim 8, further comprising an organic matter conveyor having an inlet in communication with a source of organic matter and an outlet in communication with the docking area.

15. The apparatus of claim 8, wherein the substrate collector comprises a conveyor that is adapted to transport collected substrate to a collection hopper.

16. The apparatus of claim 15, wherein the collection hopper is connected to a vacuum line that is adapted to transport the collected substrate to a processing location.

17. A gantry system for delivering organic matter to a worm bed reactor, the worm bed reactor comprising a length and a width, the gantry system comprising:
a track extending along the length of the worm bed reactor; and
a distribution gantry mounted to the track, the distribution gantry comprising:
track engagements sized to engage with and move along the track; and
an organic matter receptacle having a sidewall surrounding an open bottom that extends substantially across the width of the worm bed reactor, and a conveyor below the open bottom that extends across the open bottom, the conveyor receiving organic matter from the open bottom and moving the organic matter from within the organic matter receptacle to the worm bed reactor along the length of the worm bed reactor as the distribution gantry moves along the track.

18. The gantry system of claim 17, wherein the open bottom is adjustable between an open state and a closed state.

19. The gantry system of claim 17, wherein the conveyor is adapted to move across the open bottom at the same rate as the track engagements move along the track.

20. The gantry system of claim 17, wherein the distribution gantry comprises one or more sensors that detect one or more of the following: a volume of organic matter within the organic matter receptacle, a temperature of the worm bed reactor, a pH of the worm bed reactor, a moisture content of the worm bed reactor, and a worm concentration in the worm bed reactor.
